(19) United States
(12) Patent Application Publication
(10) Pub. No.: US 2026/0026689 A1
(43) Pub. Date: Jan. 29, 2026

(54) SELF-MEASUREMENT OPHTHALMIC INSTRUMENT

(71) Applicant: Reichert, Inc., Depew, NY (US)

(72) Inventor: Russell J. BONAVENTURA, Williamsville, NY (US)

(21) Appl. No.: 18/784,385

(22) Filed: Jul. 25, 2024

(51) Int. Cl. A61B 3/16; A61B 3/00

(52) U.S. Cl. CPC A61B 3/165; A61B 3/0008

(57) ABSTRACT

An ophthalmic instrument for self-measurement of an ophthalmic parameter has a measurement assembly operable for impinging the eye to measure the ophthalmic parameter, an occluder having one or more apertures collectively centered about a measurement axis of the measurement axis, and a source of visible light arranged to illuminate the one or more apertures. X-Y alignment of the measurement axis with the eye is indicated when the one or more apertures appear concentric with a visible portion of the measurement assembly when viewed by the eye along the measurement axis. The ophthalmic instrument may be without a focusing optical element downstream from the source of visible light, and may have an opto-electronic position detection system for automatically detecting a current X-Y-Z position of the measurement assembly as a basis for controlling illumination attributes of the source of visible light to provide self-positioning cues to a user.

### FIELD OF THE DISCLOSURE

The present disclosure relates to ophthalmic instruments which are positioned by a test subject relative to his or her own eye as a prerequisite to self-measuring an ophthalmic parameter of the eye. For example, the present disclosure relates to rebound tonometers which utilize a disposable probe for contacting a cornea of the eye to measure intraocular pressure (IOP), and non-contact tonometers which utilize an air pulse to temporarily deform the cornea to measure IOP.

### BACKGROUND OF THE DISCLOSURE

A rebound tonometer is an ophthalmic instrument that propels a movable measurement probe in a controlled manner along a measurement axis toward the cornea of an eye to measure intraocular pressure. During a measurement, the probe contacts the cornea, decelerates at a rate which depends on intraocular pressure, and then rebounds in a direction away from the cornea back toward the instrument housing. The rebound tonometer detects the motion of the measurement probe and determines intraocular pressure based on the detected motion of the probe. For example, the measurement probe may have a magnetized shaft that travels within a coil in the instrument housing. The coil may be energized momentarily to propel the probe toward the cornea by electromagnetic force, and then, after energizing current to the coil is shut off, a current may be induced in the coil by the moving probe to provide a detectable voltage signal representing velocity of the probe as a function of time. Alternatively, two coils may be provided, wherein one coil is used to propel the probe and the moving probe induces current in the other coil to provide a measurement voltage signal. The voltage signal may be recorded and processed to determine a measured IOP value.

Known rebound tonometers are designed for hand-held use by an operator to measure IOP of a test subject's eye. Proper three-dimensional positioning of the rebound tonometer relative to the eye immediately prior to measurement is an important factor for measurement accuracy and repeatability. The rebound tonometer is ideally positioned by the operator such that the measurement axis intersects the corneal apex while the test subject gazes directly along the measurement axis (X-Y alignment), and a rounded tip of the measurement probe is located at predetermined working distance (Z distance) from the corneal surface. Although ideal three-dimensional positioning is impossible to achieve due to movement of the operator's hand holding the tonometer and/or movement of the test subject, three-dimensional positioning within an acceptable tolerance range relative to the ideal position is required to obtain a reliable measurement result.

A non-contact tonometer, also referred to as an air-puff tonometer, is another type of ophthalmic instrument for measuring IOP. Like a rebound tonometer, a non-contact tonometer may be hand-held and manually positioned by an operator relative to an eye of a test subject. Non-contact tonometers have a three-dimensional positioning requirement similar to that described above for a rebound tonometer, except that a fluid discharge tube for discharging an air pulse toward the eye defines the measurement axis and working distance.

Frequent measurement of IOP is recognized as being beneficial to effectively treating glaucoma and other diseases associated with elevated IOP. However, limited patient access to a trained operator for measuring IOP is a major impediment to frequent IOP measurement. Most known positioning systems are designed to guide an operator in positioning an ophthalmic instrument relative to an eye of a patient. These position guidance systems typically have a display showing positioning cues visible from an operator side of the ophthalmic instrument opposite a patient or test subject side of the ophthalmic instrument from which a probe or air pulse is discharged toward the eye. Consequently, such position guidance systems are not suited for self-measurement.

Adding to the problem, the test subject must gaze directly at a measurement assembly of the ophthalmic instrument operable for impinging the eye to measure the ophthalmic parameter. For example, the measurement assembly may include a cylindrical barrel enclosing one or more electromagnetic coils to propel a probe toward the eye, or an air pulse discharge tube for discharging an air pulse at the eye. Consequently, the test subject's view is limited, and the test subject is not free to glance at a display elsewhere on the ophthalmic instrument for positioning cues and guidance.

An ophthalmic instrument position guidance system for self-measurement is known from US2021/0298598A1 (Salkola et al.). The system comprises an optical component (e.g., a lens) aligned on the measurement axis and having a focal power to define a focal point on the measurement axis, and multiple light sources positioned at predetermined locations relative to the measurement axis and the optical component such that multiple light rings are visible along the measurement axis only when the viewer's eye is within a predefined working distance range from the optical component. Adherence to tight dimensional tolerances is critical for proper implementation, thereby making the ophthalmic instrument technically demanding and costly to manufacture.

What is needed is a positioning system for an ophthalmic instrument that is uncomplicated to manufacture and enables a test subject to easily and accurately position the ophthalmic instrument relative to his or her own eye to administer self-measurements, for example self-measurements of IOP.

### SUMMARY OF THE DISCLOSURE

The present disclosure provides a spatially compact, lightweight ophthalmic instrument designed to be self-positioned by a user relative to his or her eye for taking an ophthalmic measurement. The ophthalmic instrument may be, for example, a rebound tonometer which propels a probe along a measurement axis of the instrument toward the eye, or a non-contact tonometer which discharges a fluid pulse along a measurement axis of the instrument toward the eye.

The ophthalmic instrument may generally comprise a measurement assembly defining the measurement axis for X-Y alignment with the eye to measure an ophthalmic parameter, an occluder having one or more apertures collectively centered about the measurement axis, and a source of visible light arranged to illuminate the one or more apertures of the occluder with visible light. X-Y alignment of the measurement axis with the eye may is indicated when the one or more apertures collectively appear concentric with a visible portion of the measurement assembly when the one or more apertures are viewed by the eye along the measurement axis. The measurement assembly is operable for impinging the eye, for example with a probe or an air pulse, to measure the ophthalmic parameter. The ophthalmic instrument may be without a focusing optical element downstream from the source of visible light.

In an embodiment of the disclosure, the visible portion of the measurement assembly may have a circular profile centered about the measurement axis when the measurement assembly is viewed along the measurement axis, and the one or more apertures may be a single circular aperture. The ophthalmic instrument may have a translucent element arranged between the source of visible light and the occluder for diffusing light emitted by the source of visible light. In one embodiment, the source of visible light comprises an array of light-emitting diodes.

The ophthalmic instrument may further comprise an opto-electronic position detection system for automatically detecting a current X-Y-Z position of the measurement assembly relative to the eye and determining a three-dimensional position difference between the current X-Y-Z position and an ideal X-Y-Z alignment position of the measurement assembly relative to the eye. The opto-electronic position detection system may be connected to the source of visible light by way of an illumination control system, and a color of the visible light illuminating the one or more apertures may be set by the illumination control system based on the three-dimensional position difference between the current X-Y-Z position and the ideal X-Y-Z alignment position. In this way, visual positioning cues may be given to the user to facilitate self-positioning. Similarly, the opto-electronic position detection system may be connected to an audio speaker for generating audible positioning cues. Information from the opto-electronic position detection system may enable a controller of the ophthalmic instrument to determine when proper positioning is achieved and automatically command operation of the measurement assembly to take a measurement.

In one embodiment, the ophthalmic instrument may comprise a tilt sensing system for detecting if a current tilt angle of the measurement axis relative to horizontal is greater than a predetermined threshold tilt angle. The tilt sensing system may be connected to the source of visible light by way of the illumination control system, and the illumination control system may control the source of visible light to provide a corresponding visual cue to the user, for example by causing the source to blink on-and-off while the current tilt angle of the measurement axis is greater than the predetermined threshold tilt angle.

### BRIEF DESCRIPTION OF THE DRAWING VIEWS

The nature and mode of operation of the present invention will now be more fully described in the following detailed description taken with the accompanying drawing figures, in which:

FIG. 1 is a perspective view of an ophthalmic instrument formed in accordance with an embodiment of the present disclosure;

FIG. 2 is cross-sectional perspective view showing a measurement head of the ophthalmic instrument;

FIG. 3 is another perspective view showing components of a positioning system of the ophthalmic instrument within the measurement head;

FIG. 4 is a plan view of a front plate of the measurement head;

FIG. 5A is a schematic top view illustrating components of an opto-electronic position detection system of the ophthalmic instrument in relation to an eye of a test subject;

FIG. 5B is a schematic side view illustrating components of the position detection system in relation to an eye of a test subject;

FIG. 6 is a schematic view of an area detector of the position detection system;

FIG. 7 is a schematic electronic block diagram of the ophthalmic instrument;

FIG. 8 is an example of a calibration image captured by the area detector of the positioning system during a calibration procedure;

FIGS. 9A, 9B, and 9C show examples of positioning images captured by the area detector during a measurement procedure;

FIGS. 10A-10E illustrate views of the measurement head as it appears to an eye of a test subject under various positioning conditions of the ophthalmic instrument relative to the eye;

FIG. 11 is a logic table illustrating audio and visual cues provided to a test subject to guide the test subject in positioning the ophthalmic instrument in three dimensions relative to an eye of the test subject; and

FIG. 12 is a flow chart illustrating operation of the ophthalmic instrument to measure an ophthalmic parameter of an eye.

### DETAILED DESCRIPTION OF THE INVENTION

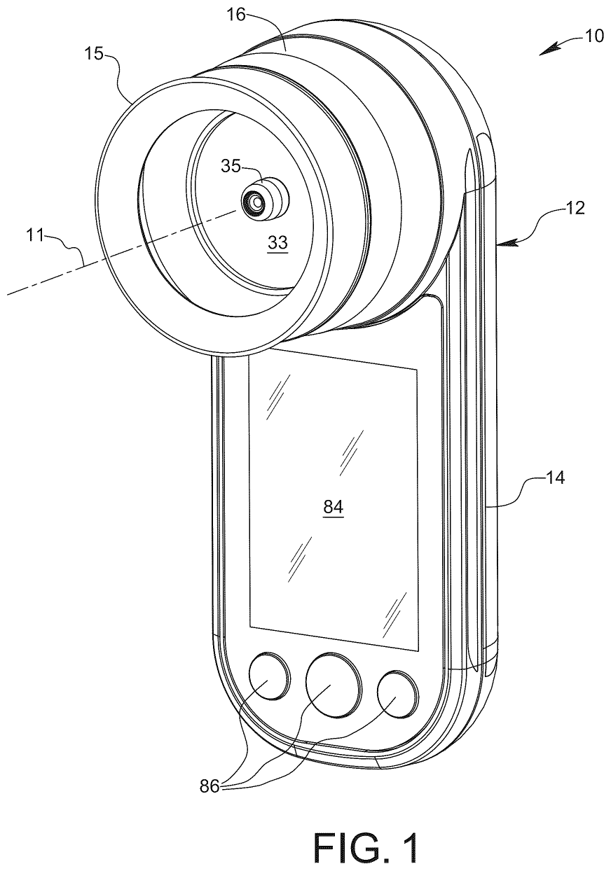

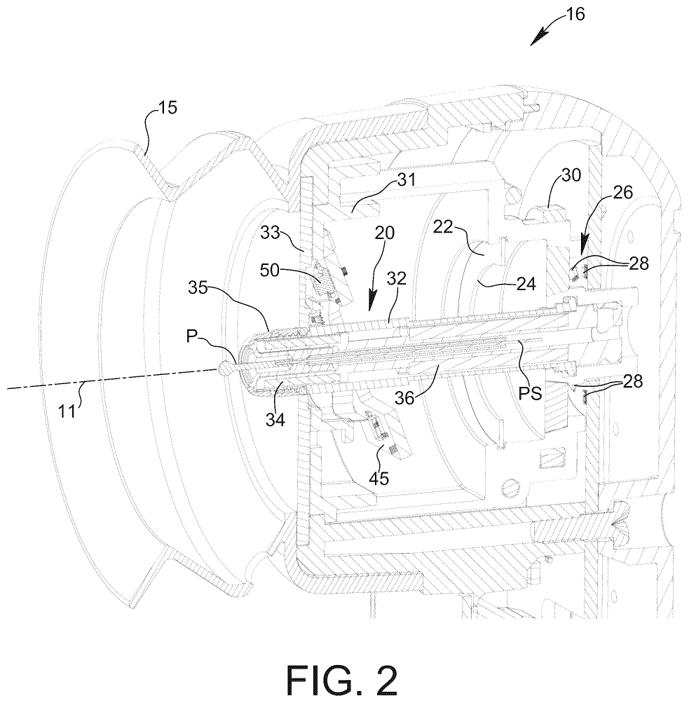

FIGS. 1 and 2 show an ophthalmic instrument 10 for measuring an ophthalmic parameter of an eye in accordance with an embodiment of the present disclosure. In the figures, ophthalmic instrument 10 is embodied as a rebound tonometer for measuring IOP, however it is understood that ophthalmic instrument 10 may be embodied as a non-contact tonometer for measuring IOP, or may be embodied as another type of ophthalmic instrument for measuring a parameter of the eye other than IOP. Ophthalmic instrument 10 comprises a measurement axis 11. In the context of the illustrated rebound tonometer, measurement axis 11 is an axis along which a measurement probe P is propelled toward an eye of a test subject. In the context of a non-contact tonometer (not shown), measurement axis 11 is an axis of a fluid discharge tube through which a fluid pulse, e.g. an air puff, is directed at an eye of a test subject. Ophthalmic instrument 10 may have a housing 12 defining a handle portion 14 and a measurement head 16 atop handle portion 14, wherein the measurement axis 11 extends through the measurement head 16.

Ophthalmic instrument 10 may also comprise an eye cup 15 at a front end of measurement head 16. Eye cup 15 may contact the test subject around the eye (e.g., cheekbone, forehead, or other facial area) when positioning measurement head 16 near the eye for measurement. In some embodiments, eye cup 15 may be made of a deformable, elastomeric material and may be configured similar to a compressible bellows, whereby the eye cup may confirm to a test subject's face around an eye of the test subject while permitting position adjustment of measurement head 16 relative to the enclosed eye in an X direction (left-right), a Y direction (up-down), and a Z direction (toward-away from eye). In some embodiments, eye cup 15 may be removably mounted on measurement head 16, and in other embodiments, eye cup 15 may be provided as a permanent part of measurement head 16.

In the illustrated embodiment, measurement head 16 includes a measurement assembly 20 operable for impinging the eye to measure an ophthalmic parameter, such as IOP and/or a biomechanical property of the cornea such as corneal hysteresis. In the present disclosure, “impinging the eye” means applying an external influence to the eye either directly or indirectly, including but not limited to propelling a probe to contact and rebound from the eye and discharging a fluid pulse to interact with the eye. Measurement assembly 20 defines measurement axis 11 for X-Y alignment with an eye to measure the ophthalmic parameter.

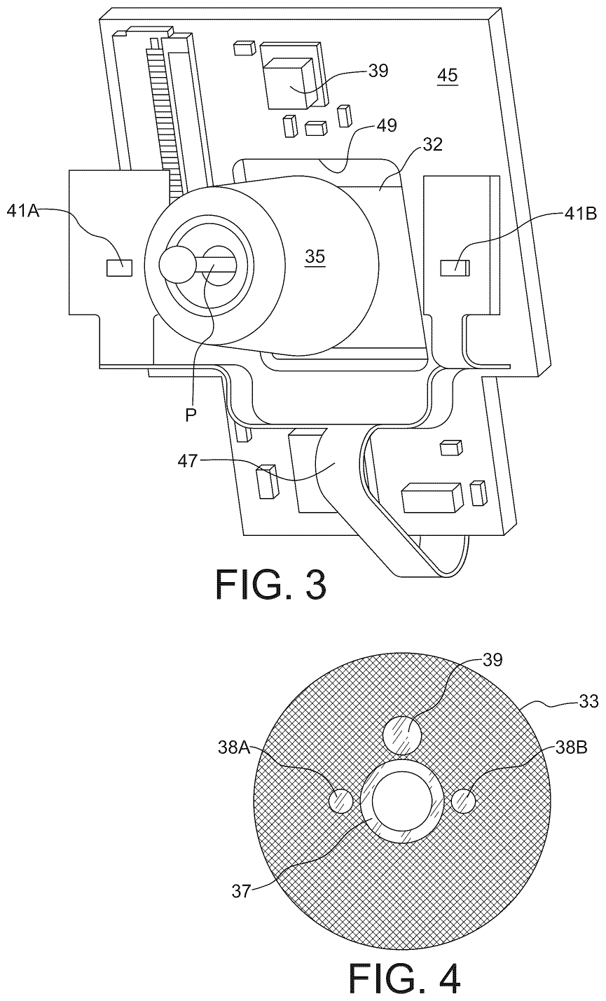

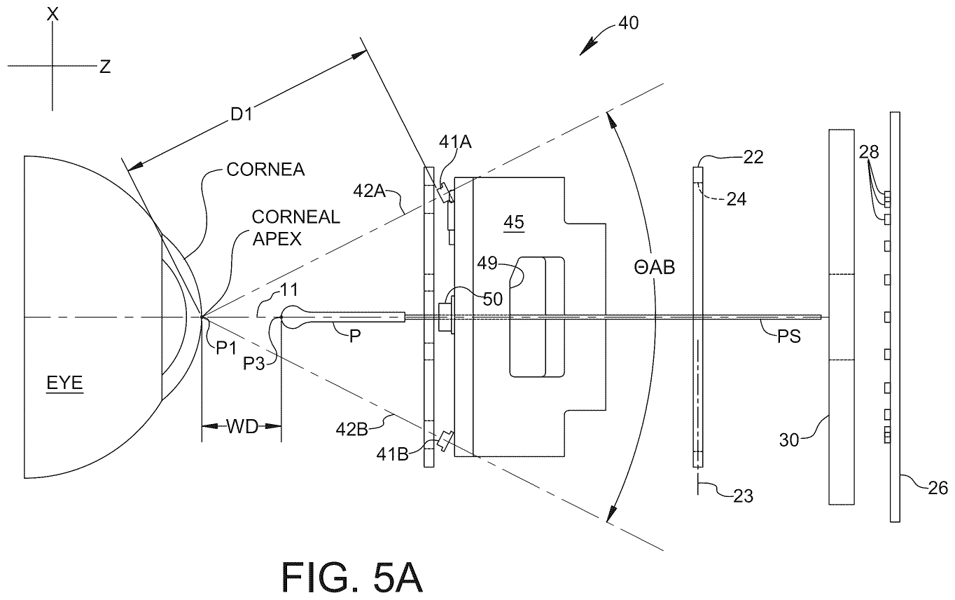

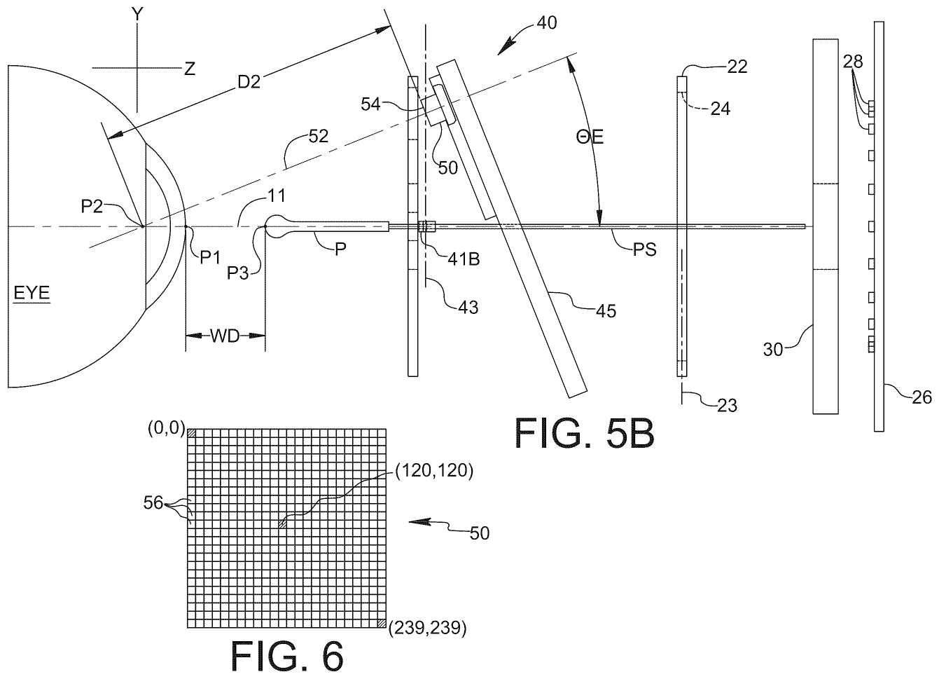

As may be seen in FIG. 2 through FIG. 5B, measurement head 16 also includes an occluder 22 having one or more apertures 24 collectively centered about measurement axis 11, and a source of visible light 26 arranged to illuminate the one or more apertures 24 with visible light. In the embodiment shown in the figures, the source of visible light 26 comprises a circular array (i.e., a ring array) of light-emitting diodes 28 centered about measurement axis 11 and occluder 22 has exactly one circular aperture 24 centered about measurement axis 11 such that visible light from light-emitting diodes 28 illuminates aperture 24. Alternatively, the source of visible light 26 may be a single light-emitting diode coupled with a waveguide or a diffusing element to form an illumination source centered about measurement axis 11. Alternatively, occluder 22 may have a plurality of apertures 24 centered about measurement axis 11, for example a circular array of apertures 24 centered and angularly spaced about the measurement axis. As best seen in FIGS. 4 and 5, occluder 22 may be arranged such that the one or more apertures 24 are in a plane 23 normal to measurement axis 11. As shown in the illustrated embodiment, measurement head 16 may have a translucent element 30 arranged between source of visible light 26 and occluder 22 for diffusing light emitted by the source of visible light before the light illuminates the one or more apertures 24 of the occluder.

If ophthalmic instrument 10 is embodied as a rebound tonometer, then measurement assembly 20 may include a barrel 32 enclosing one or more electromagnetic coils 34, 36, wherein the measurement assembly receives probe P and propels the probe along the measurement axis toward the eye to impinge the eye and measure the ophthalmic parameter of the eye. In the illustrated embodiment, measurement probe P has a magnetized shaft PS coaxially received in front coil 34 and rear coil 36 in barrel 32. Front coil 34 may be energized momentarily to propel probe P toward the cornea by electromagnetic force. Rear coil 36 may serve as a sensing coil for sensing motion of probe P, wherein current is induced in rear coil 36 to provide a detectable voltage signal representing velocity of probe P as a function of time. The voltage signal may be recorded and processed in a known manner to determine a measured IOP value. Barrel 32 may protrude from housing 12 though aligned circular openings in a support frame 31 and a front plate 33 of measurement head 16. Barrel 32 may have a cap 35 surrounding a protruding front portion of the barrel. As shown in FIG. 4, front plate 33 may be a transparent plate having a rear surface coated by an opaque coating except for regions where transmission of light through the front plate is needed. One such region is an annular light-transmitting window 37 surrounding barrel 32.

If ophthalmic instrument 10 is embodied as an NCT, then measurement assembly 20 may include an air pulse discharge tube (not shown) in place of barrel 32, wherein the air pulse discharge tube discharges an air pulse along measurement axis 11 toward the eye to impinge the eye and measure an ophthalmic parameter of the eye.

As will be described in further detail below, a visible portion of measurement assembly 20 is centered about measurement axis 11 when the measurement assembly is viewed by the eye along the measurement axis. Consequently, X-Y alignment of measurement axis 11 with the eye is indicated when the one or more illuminated apertures 24 collectively appear concentric with the visible portion of measurement assembly 20 when the one or more illuminated apertures 24 are viewed by the eye along the measurement axis. Where the visible portion of measurement assembly 20 is defined by a cylindrical or frusto-conical barrel or air pulse discharge tube, the visible portion of the measurement assembly will have a circular profile centered about measurement axis 11 when measurement assembly 20 is viewed along measurement axis 11.

In an aspect of the present disclosure, ophthalmic instrument 10 is without a focusing optical element downstream from the source of visible light 26 (i.e., after the source in a direction of propagation of light emitted by the source), thereby saving cost related to parts and assembly needed to supply and mount such an optical element and avoiding variations related to focusing power tolerances of such an optical element.

Ophthalmic instrument 10 may further comprise an opto-electronic position detection system generally identified by reference numeral 40 in FIGS. 5A and 5B. Position detection system 40 includes a first light source 41A spaced apart from measurement axis 11 and arranged to direct a first illumination beam along a first illumination axis 42A, and a second light source 41B spaced apart from measurement axis 11 and arranged to direct a second illumination beam along a second illumination axis 42B. Position detection system 40 further includes an area detector 50 spaced apart from measurement axis 11 and from the first and second light sources 41A, 41B. Area detector 50 has an observation axis 52 extending normal to a detection plane 54 of the area detector in which a two-dimensional array of photosensitive pixels 56, shown in FIG. 6, is arranged. First illumination axis 42A, second illumination axis 42B, and observation axis 52 extend respectively through light-transmitting windows 38A, 38B, and 39 located adjacent annular window 37 at uncoated regions of front plate 33. As will be described in greater detail below, area detector 50 is configured and arranged to continually capture positioning images of an eye when ophthalmic instrument 10 is positioned near the eye in preparation for a measurement.

By way of non-limiting example, each of first and second light sources 41A, 41B may be a light-emitting diode (LED) which emits red diffuse light. Use of red LEDs is advantageous because it allows the positioning system to distinguish between light associated with the positioning system and all other colors detected by area detector 50. Also by way of non-limiting example, area detector 50 may be a 640×480 color pixel image sensing array provided as part of a CameraCubeChip™ available from OmniVision of Santa Clara, California under Part Number OVM7692-RYAA.

The figures illustrate one possible implementation of position detection system 40 in which area detector 50 is mounted on a circuit board 45, and light sources 41A, 41B are mounted on respective branches of a bifurcated flexible connector 47 extending from circuit board 45. As best seen in FIG. 5, circuit board 45 may be inclined with respect to measurement axis 11 such that observation axis 52 of area detector 50 forms an elevation angle ⊖E with measurement axis 11. Circuit board 45 may include an opening 49 through which barrel 32 may extend. In the depicted implementation, first illumination axis 42A and second illumination axis 42B are coplanar with measurement axis 11 in a horizontal plane and form an angle ⊖AB which is bisected by measurement axis 11. According to the illustrated implementation, observation axis 52 may be coplanar with measurement axis 11 in a vertical plane. First illumination axis 42A and second illumination axis 42B may converge at a first point P1 along measurement axis 11 within a field of view of area detector 50, and observation axis 52 may intersect measurement axis 11 at a second point P2 along measurement axis 11 spaced from first point P1. Ophthalmic instrument 10 may further comprise a working distance reference point P3 on measurement axis 11, wherein a working distance WD of instrument 10 relative to the eye is defined as the distance between reference point P3 and the corneal surface of the eye. For example, where ophthalmic instrument 10 is a rebound tonometer, working distance reference point P3 may be a point at a front tip of measurement probe P, and where ophthalmic instrument 10 is a non-contact tonometer, working distance reference point P3 may be a point at a front tip of a fluid discharge tube of the tonometer. In the illustrated implementation, a distance between reference point P3 and second point P2 is greater than a distance between reference point P3 and first point P1. As best understood from FIGS. 5A and 5B, first light source 41A, second light source 41B, and area detector 50 may be arranged such that they are intersected by a plane 43 which is normal to measurement axis 11.

With regard to the illustrated implementation, the following dimensions may be used in practice, however these dimensions are provided merely as examples and with the understanding that other dimensions may be used in practice: working distance WD=6.00 mm; illumination axis angle ⊖AB=53.6°; observation axis elevation angle ⊖E=21.8°; illumination distance D1=20.012 mm (for both light sources 41A and 41B); and observation distance D2=23.07 mm.

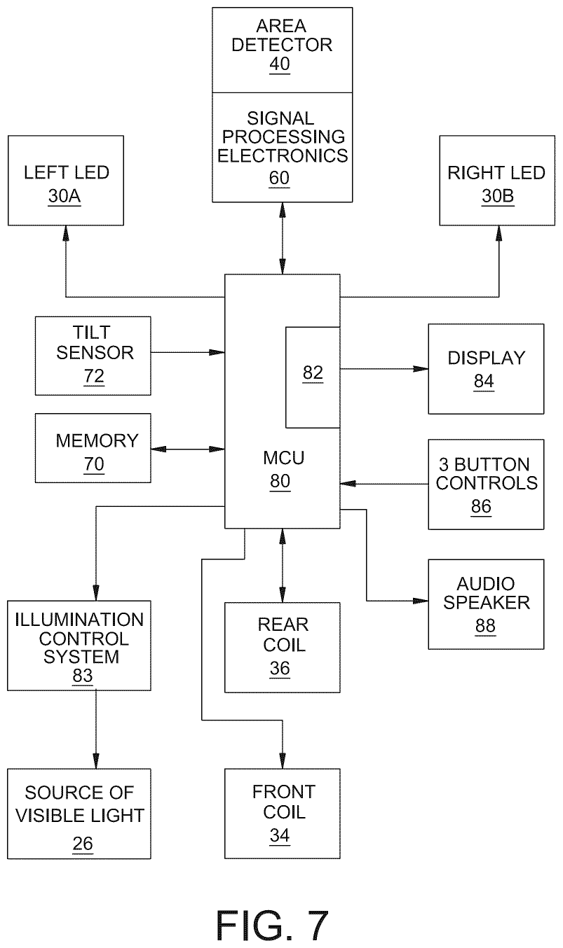

FIG. 7 is a schematic electronic block diagram of ophthalmic instrument 10. Ophthalmic instrument 10 comprises signal processing electronics 60 connected to area detector 50 for receiving the plurality of pixel signals and converting the plurality of pixel signals to a digital positioning image. For example, signal processing electronics 60 may be embodied by on-board signal processing electronics provided as part of the aforementioned CameraCubeChip™, which includes an analog-to-digital converter, a digital signal processor and formatter, and an image output interface.

Ophthalmic instrument 10 also comprises a memory 70 which stores positioning calibration information. For example, memory 70 may be embodied as a nonvolatile memory which retains stored information when power to ophthalmic instrument 10 is shut off. Memory 70 may be a ferroelectric random access memory (F-RAM) module or another type of memory. An example of a suitable F-RAM module is available from Cypress Semiconductor Corporation under Part Number FM24V10GTR.

Ophthalmic instrument 10 comprises a microcontroller 80 programmed by software instructions stored in memory to control various operating functions of ophthalmic instrument 10. By way of non-limiting example, microcontroller 80 may be embodied as a microcontroller available from STMicroelectronics under Part Number STM32LAR9AII6, which has an ARM® CORTEX®-M4 core as the central processing unit (CPU) and embedded Flash memory. Alternatively, microcontroller 80 may be embodied as another type of controller configured to control operating functions of ophthalmic instrument 10.

Ophthalmic instrument 10 further comprises an image evaluation module 82 configured by stored software instructions to evaluate the digital positioning images which are output from signal processing electronics 60 with reference to the positioning calibration information stored in memory 70, and to compute a position difference representing a difference between a current three-dimensional position of ophthalmic instrument 10 relative to the eye and an ideal three-dimensional position of the ophthalmic instrument 10 relative to the eye. As illustrated in FIG. 7, image evaluation module 82 may be incorporated in microcontroller 80. Alternatively, image evaluation module 82 may be embodied by a separate computational circuit module connected to microcontroller 80.

Ophthalmic instrument 10 may also comprise an illumination control system 83 connected to microcontroller 80 and source of visible light 26 for controlling the source of visible light with regard to one or more illumination parameters such as color, blinking status (i.e., whether illumination is continuous or blinking on and off), blinking frequency, and brightness.

Ophthalmic instrument 10 also comprises a display 84 connected to microcontroller 80 for presenting information to a test subject using ophthalmic instrument 10 for self-measurement. For example, display 84 may be used to display measurement results and other measurement data to the user. By way of non-limiting example, display 84 may be a liquid crystal display (LCD).

Ophthalmic instrument 10 may further comprise menu navigation/selection buttons 86 connected to microcontroller 80 and enabling a user to set operating parameters of ophthalmic instrument 10 and perform functions in conjunction with operating system menus displayed on display 84. Ophthalmic instrument 10 may also comprise an audio speaker 88 connected to microcontroller 80.

Ophthalmic instrument 10 may further comprise a tilt sensor 72 connected to microcontroller 80 to provide a tilt sensing system for determining if a current tilt angle of measurement axis 11 relative to horizontal is greater than a predetermined threshold tilt angle. Tilt sensor 72 may be, for example, a Microelectronics sensor part number LIS2DW12 that generates a tilt signal representing a detected tilt angle of measurement axis 11 relative to horizontal. Microcontroller 80 may be programmed to evaluate the tilt signal sent by tilt sensor 72 by comparing the detected tilt angle to a predetermined threshold tilt angle stored in memory 70 to determine if the detected tilt angle is greater than the predetermined threshold tilt angle. The tilt sensing system embodied by tilt sensor 72 and microcontroller 80 may be connected to illumination control system 83 and audio speaker 88 as shown in FIG. 7.

As described above, area detector 50 continually captures positioning images of an eye when ophthalmic instrument 10 is positioned near the eye in preparation for a measurement, and the captured positioning images are converted from analog to digital format by signal processing electronics 60. The digital positioning images are evaluated by image evaluation module 82 to provide information indicating the positioning status of ophthalmic instrument 10 relative to the eye in three dimensions X, Y, and Z.

To enable image evaluation, ophthalmic instrument 10 is calibrated using a false “calibration eye,” for example a false eye made of glass, to capture at least one calibration image from which positioning calibration information is determined and stored in memory 70. The calibration eye may be, for example, a glass spherical ball having a radius of eight millimeters, which is approximately the average radius of curvature of an anterior surface of a human cornea. The calibration image is captured when ophthalmic instrument 10 is at an ideal three-dimensional position relative to the calibration eye for carrying out a measurement. For example, the ideal three-dimensional position may correspond to a condition in which measurement axis 11 intersects the corneal apex and is substantially perpendicular to the local corneal surface at the corneal apex, and the tip point P3 of probe P is at a predetermined working distance WD away from the corneal surface. In a current embodiment, the predetermined working distance WD may be six millimeters, however the predetermined working distance WD may be another value. Where ophthalmic instrument 10 is a rebound tonometer, calibration may be carried out using a calibration tool which incorporates the calibration eye and mounts directly in ophthalmic instrument 10 along measurement axis 11 in place of a probe P. For example, the calibration eye may be mounted at an end of a shaft which is similar to probe shaft PS whereby the calibration tool shaft may be releasably and coaxially retained in front coil 34 and rear coil 36 of ophthalmic instrument 10. The calibration tool may be configured so that when the calibration tool is mounted in ophthalmic instrument 10, the calibration eye is located such that an apex of the calibration eye is intersected by measurement axis 11 at a Z-axis position corresponding to the predetermined working distance WD from the front tip point P3 of a probe P if a probe P were mounted in ophthalmic instrument 10 instead of the calibration tool. Where ophthalmic instrument 10 is a non-contact tonometer, the calibration tool may have a mounting shaft sized for slidable receipt within the axial passage of the fluid pulse (e.g., air puff) discharge tube to align the calibration eye on measurement axis 11 at an ideal Z-axis working distance from an exit end of the discharge tube for purposes of calibration.

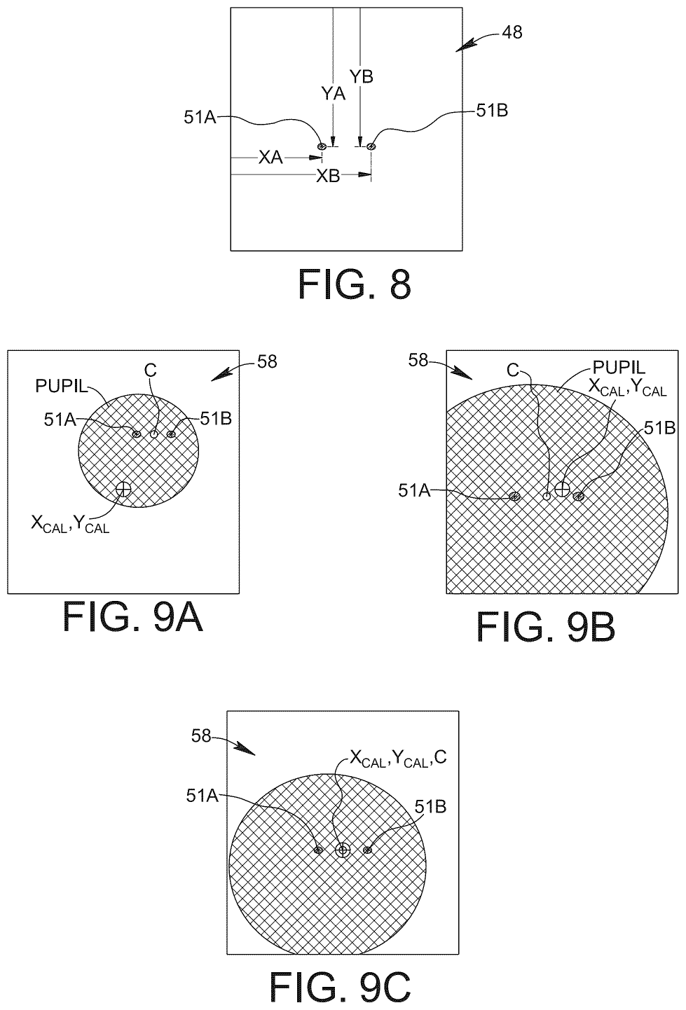

FIG. 8 shows an example of a calibration image 48 captured by area detector 50 when ophthalmic instrument 10 is at the predetermined ideal three-dimensional measurement position relative to a calibration eye, and digitized by signal processing electronics 60. Calibration image 48 represents a reflected-light image of a facing surface region of the calibration eye when first light source 41A and second light source 41B are illuminated. Consequently, calibration image 48 includes a first source image 51A corresponding to first light source 41A and a second source image 51B corresponding to second light source 41B. Image evaluation module 82 may be programmed to evaluate calibration image 48 to determine location coordinates XA, YA of first source image 51A and location coordinates XB, YB of second source image 51B, wherein the location coordinates represent a location of the corresponding source image in the two-dimensional sensing surface of area detector 50. For example, the coordinate values may be based on pixel values counted relative to an origin corner of the sensing surface of area detector 50, as illustrated in FIG. 6. Evaluation of calibration image 48 to determine the locations of source images 51A, 51B may involve finding the pixel groups registering the highest intensity for a specific color corresponding to the color of light sources 41A, 41B. For example, where area detector 50 is a color Red-Green-Blue (RGB) sensor and light sources 41A, 41B are red LEDs, the red, green, and blue pixel color separation may be analyzed to differentiate the red LED reflections defining source images 51A, 51B from the rest of calibration image 48. Exclusion criteria may be applied to the location, size, intensity, and/or separation of source images 51A, 51B to prevent false location detection. The respective centroids of source images 51A, 51B may be calculated to determine the corresponding two-dimensional locations (XA, YA) and (XB, YB) of source images 51A, 51B.

The two-dimensional locations (XA, YA) and (XB, YB) of source images 51A, 51B in calibration image 48 may be stored directly in memory 70 and/or used to calculate a three-dimensional calibration position coordinate (X_{CAL}, Y_{CAL}, Z_{CAL}) stored in memory 70. For example, the three-dimensional calibration position coordinate (X_{CAL}, Y_{CAL}, Z_{CAL}) may be calculated as follows. X_{CAL}, which corresponds to an ideal horizontal left-right position of ophthalmic instrument 10 relative to an eye, may be calculated as the average X location of the two source images 51A, 51B in calibration image 48:

$${X}_{CAL} = ( XA + XB ) / 2$$

Y_{CAL}, which corresponds to an ideal vertical up-down position of ophthalmic instrument 10 relative to an eye, may be calculated as the average Y location of the two source images 51A, 51B in calibration image 48:

$${Y}_{CAL} = ( YA + YB ) / 2$$

Z_{CAL}, which corresponds to an ideal working distance position of ophthalmic instrument 10 along measurement axis 11 relative to an eye, may be calculated as the horizontal spacing between the two source images 51A, 51B in calibration image 48:

$${Z}_{CAL} = XB - XA$$

As may be understood, the horizontal spacing between source images 51A, 51B is inversely proportional to the working distance. In other words, as the working distance of ophthalmic instrument 10 from the eye is decreased, the horizontal spacing between source images 51A, 51B will increase, and as the working distance of ophthalmic instrument 10 from the eye is increased, the horizontal spacing between source images 51A, 51B will decrease.

The two-dimensional locations (XA, YA) and (XB, YB) of source images 51A, 51B, and the three-dimensional calibration position coordinate (X_{CAL}, Y_{CAL}, Z_{CAL}), may individually and/or collectively be considered “positioning calibration information.” The positioning calibration information determined through calibration of ophthalmic instrument 10 as described above may be stored in memory 70 for later use during a normal (i.e. non-calibration) measurement procedure to determine when ophthalmic instrument 10 is near enough to the ideal three-dimensional measurement position to allow a measurement to be initiated.

FIGS. 9A, 9B, and 9C illustrate examples of positioning images 58 captured by area detector 50 during a typical measurement procedure. As with calibration image 48, area detector 50 generates a plurality of pixel signals collectively representing each positioning image, and the pixel signals are digitized by signal processing electronics 60 to provide a digital positioning image 58. In the examples shown in FIGS. 9A, 9B, and 9C, a portion of the eye to be measured, including the pupil of the eye, is within a field of view of area detector 50 and is visible in positioning image 58. Similar to calibration image 48, each positioning image 58 may include a first source image 51A corresponding to first light source 41A and a second source image 51B corresponding to second light source 41B. For sake of understanding, a centroid C mid-way between source images 51A, 51B and the ideal alignment location X_{CAL}, Y_{CAL} determined by calibration are also shown, although these points do not appear in the actual positioning image 58. In the example of FIG. 9A, the centroid C between source images 51A, 51B is located above and to the right of ideal alignment location X_{CAL}, Y_{CAL} in positioning image 58, which corresponds to a positioning state in which measurement axis 11 of instrument 10 is below and to the left of the corneal apex and instrument 10 must be moved upward and to the right for proper measurement. As another example, in FIG. 9B, the centroid C of source images 51A, 51B is located slightly below and to the left of ideal alignment location X_{CAL}, Y_{CAL} in positioning image 58, which corresponds to a positioning state in which measurement axis 11 of instrument 10 is just above and to the right of the corneal apex, and instrument 10 must be moved slightly downward and to the left for proper measurement. In the example of FIG. 9C, the centroid C of source images 51A, 51B and the ideal alignment location X_{CAL}, Y_{CAL} are at approximately the same location in positioning image 58, indicating that instrument 10 is approximately at the ideal X-Y alignment position for measurement wherein measurement axis 11 intersects the corneal apex.

As explained above with respect to calibration image 48, the horizontal spacing between source images 51A, 51B in positioning image 58 is inversely proportional to the working distance WD. For example, FIG. 9A illustrates a positioning state in which the working distance is too far from the eye, FIG. 9B illustrates a positioning state in which the working distance is too close to the eye, and FIG. 9C illustrates a positioning state in which the working distance is within an acceptable tolerance for proper measurement. The current three-dimensional position coordinate (X, Y, Z) may be calculated from positioning image 58 the same way the three-dimensional calibration position coordinate (X_{CAL}, Y_{CAL}, Z_{CAL}) is calculated from calibration image 48, as described above.

As mentioned above, image evaluation module 82 may be configured by stored software instructions to compute a position difference representing a difference between the current three-dimensional position of ophthalmic instrument 10 represented by coordinate (X, Y, Z) and the ideal three-dimensional position of ophthalmic instrument 10 represented by coordinate calibration coordinate (X_{CAL}, Y_{CAL}, Z_{CAL}). For example, a position difference (ΔX, ΔY, ΔZ) may be calculated as follows:

$$Δ X = X - {X}_{CAL} ,$$

$$Δ Z = Z - {Z}_{CAL} , and$$

$$Δ Y = Y - {Y}_{CAL} - Δ Z .$$

In the calculation of ΔY, subtracting ΔZ compensates for the change in camera angle (i.e., the change in the elevation angle ⊖E of observation axis 52) as the Z distance from the eye changes. Proper positioning of instrument 10 relative to the eye for measurement purposes may be judged by whether or not the computed position difference is within a predetermined positioning tolerance for measurement purposes. For example, to be within the predetermined positioning tolerance, the current X, Y pixel position of instrument 10 must be within a predetermined radius of the pixel location X_{CAL}, Y_{CAL}, and an absolute value of ΔZ must be less than or equal to a predetermined Z axis tolerance expressed in pixels. In spatial coordinates, it has been found that measurement axis 11 is preferably within a radius of 1 mm from the corneal apex in the X and Y positioning directions, and within +1.5 mm of the ideal working distance in the Z positioning direction, however other values may be used. Instead of requiring the current X, Y pixel position of instrument 10 to be within a predetermined radius of the pixel location X_{CAL}, Y_{CAL}, a bounding box centered around pixel location X_{CAL}, Y_{CAL} may be defined whereby each of ΔX and ΔY must be within its own respective tolerance.

As ophthalmic instrument 10 is being positioned relative to the eye in preparation for a measurement, area detector 50 generates a plurality of pixel signals collectively representing a current positioning image of the field of view of area detector 50. The pixel signals are digitized by signal processing electronics 60 to provide a corresponding digital positioning image 58. The current digital positioning image 58 is evaluated by image evaluation module 82 as described above such that the current positioning status of ophthalmic instrument 10 is known as ophthalmic instrument 10 is being positioned relative to the eye for a measurement. More specifically, image evaluation module 82 is programmed as described above to determine if the current position differences ΔX, ΔY, and ΔZ values are respectively within predetermined tolerance ranges to signify proper positioning for measurement.

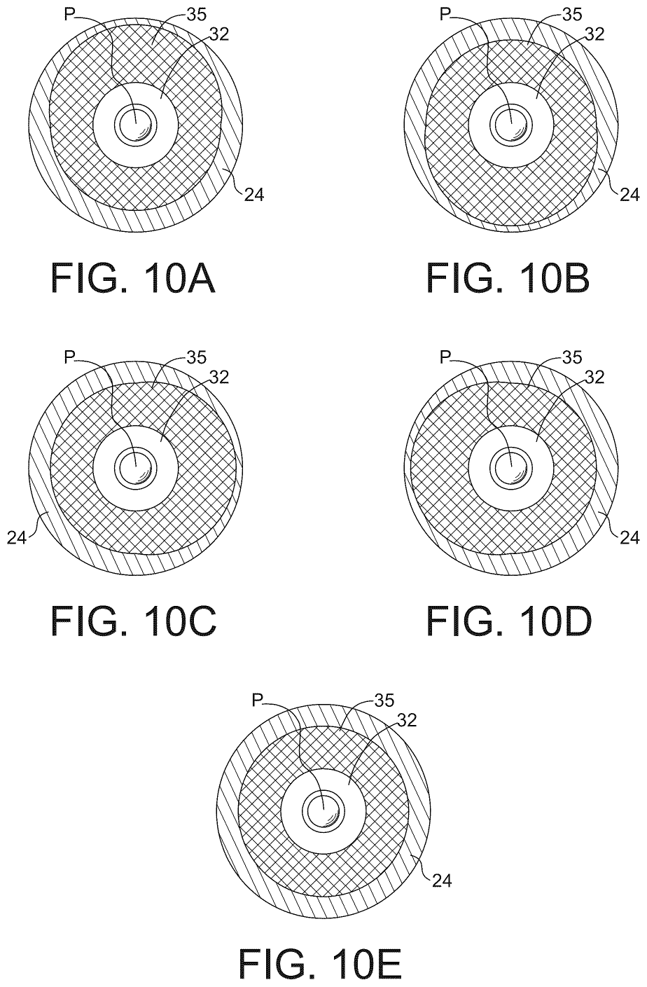

FIGS. 10A-10E illustrate views of measurement head 16 as it appears to an eye of a test subject under various positioning conditions of ophthalmic instrument 10 relative to the eye during a typical measurement procedure. During positioning, the test subject is manually trying to correctly position ophthalmic instrument 10 in three dimensions for taking a measurement and is simultaneously viewing measurement assembly 20 generally along a direction of measurement axis 11. Thus, the test subject sees the tip of probe P surrounded by a front end face of barrel 32, cap 35, and the one or more illuminated apertures 24. In the illustrated embodiment, a single illuminated aperture 24 is provided and appears as a radially outer ring surrounding cap 35. In the view of FIG. 10A, measurement head 16 is too high relative to the test subject's eye and illuminated aperture 24 does not appear concentric with cap 35 and the front end face of barrel 32. In the view of FIG. 10B, measurement head 16 is too low relative to the test subject's eye and again illuminated aperture 24 does not appear concentric with cap 35 and the front end face of barrel 32. In the views of FIG. 10C and FIG. 10D, measurement head 16 is too far right and too far left from the eye, respectively, and concentricity is not apparent. The view of FIG. 10E illustrates proper X-Y alignment of measurement head 16 with the eye wherein measurement axis 11 intersects the corneal apex and is substantially perpendicular to the local corneal surface at the corneal apex. As may be seen, illuminated aperture 24 appears concentric with cap 35 and the front end face of barrel 32, thereby indicating proper X-Y alignment to the test subject.

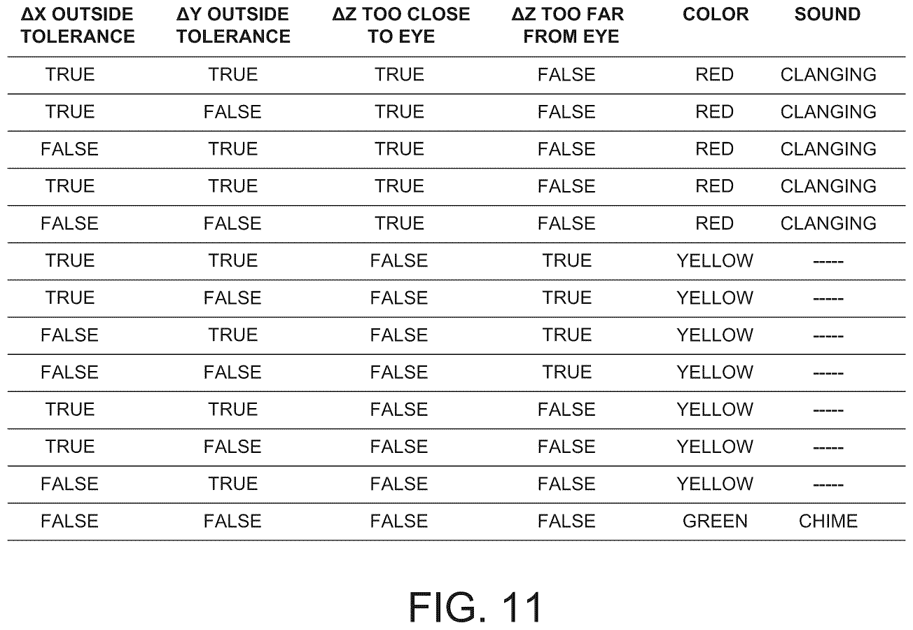

In an aspect of the present disclosure, the compliance or non-compliance with the positioning tolerances as ophthalmic instrument 10 is being positioned relative to the eye as determined by image evaluation module 82 may be used as a basis for generating further positioning cues to the test subject beyond the visual concentricity cues described in the preceding paragraph. For example, a color and/or a blinking behavior of the source of visible light 26 and/or an audible signal generated by audio speaker 88 may be based on compliance or non-compliance with positioning tolerances. FIG. 11 is a logic table illustrating examples of audio and visual cues that may be provided to a test subject to guide the test subject in positioning the ophthalmic instrument in three dimensions relative to the test subject's eye. A positioning state wherein measurement head 16 is too close to the eye is considered dangerous because the test subject may inadvertently make contact with the eye or face. Therefore, if ΔZ is outside the tolerance range in a direction indicating measurement head 16 is too close to the eye, then microcontroller 80 may be programmed to send a signal to illumination control system 83 causing the illumination control system to set the color of the source of visible light 26 to a first color, for example red, and to send a signal to audio speaker 88 to generate a first sound cue, for example a clanging sound or a crashing sound. If one or more of the positional tolerances are not met, and measurement head 16 is not too close to the eye, then microcontroller 80 may be programmed to send a signal to illumination control system 83 causing the illumination control system to set the color of the source of visible light 26 to a second color different from the first color, for example yellow, and to silence audio speaker 88. Finally, if all positional tolerances are met, then microcontroller 80 may be programmed to send a signal to illumination control system 83 causing the illumination control system to set the color of the source of visible light 26 to a third color different from the first and second colors, for example green, and to send a signal to audio speaker 88 to generate a second sound cue different from the first sound cue, for example a chime sound.

While not shown in the logic table of FIG. 11, if the eye is outside a field of view of the position detection system 40 such that the position detection system is unable to detect the eye, then the color of the visible light illuminating the one or more apertures may be set to a fourth color different from the first, second, and third colors, for example white.

If the tilt sensing system embodiment by tilt sensor 72 and microcontroller 80 determines that a current tilt angle of measurement axis 11 is greater than the predetermined threshold tilt angle, then microcontroller 80 may be programmed to send a signal to illumination control system 83 causing the illumination control system to control source of visible light 26 such that it blinks on-and-off until the current tilt angle of measurement axis 11 no longer exceeds the predetermined threshold tilt angle. Alternatively or additionally, microcontroller 80 may be programmed to send a signal to audio speaker 88 to generate a sound cue while the current detected tilt angle of measurement axis 11 is greater than the predetermined threshold tilt angle. These additional one or more cues associated with tilt angle will help the test subject manually position and orient ophthalmic instrument 10 relative to the eye.

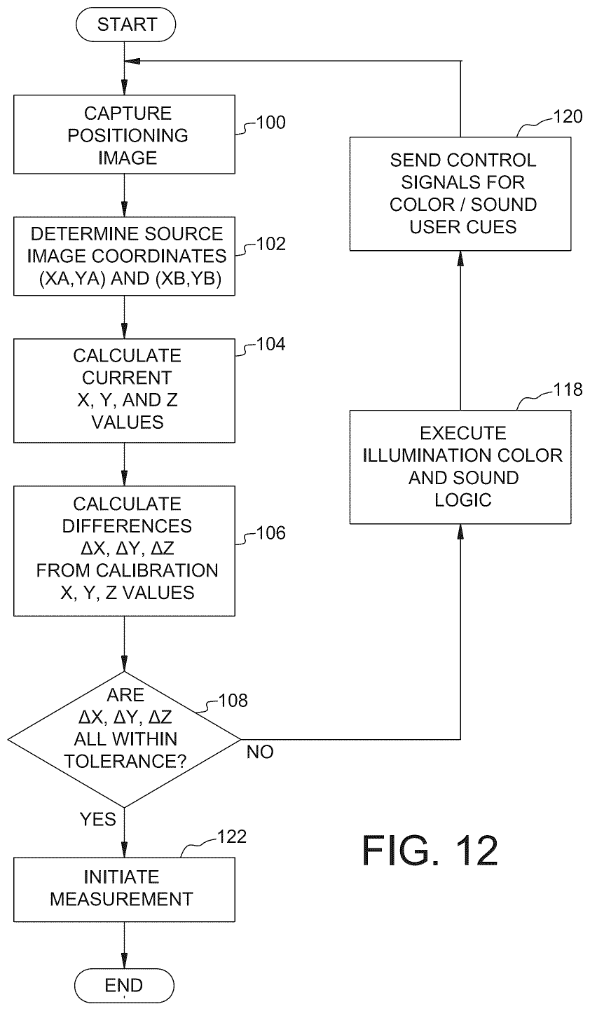

Reference is now made to FIG. 12 to describe operation of ophthalmic instrument 10 to measure an ophthalmic parameter of an eye. In step 100, a positioning image 58 is captured by area detector 50. In steps 102, 104, and 106, the source image coordinates (XA, YA) and (XB, YB) are determined, the current positioning coordinate (X, Y, Z) is calculated, and the position difference (ΔX, ΔY, ΔZ) is calculated. These steps are described in detail above. A decision block 108 then determines whether or not the computed position difference is within a predetermined positioning tolerance for measurement purposes. If not, flow branches to step 118 in which logic for controlling the color of visible light emitted by source 26 and the sound generated by audio speaker 88 is executed by microcontroller 80, for example in accordance with the logic table of FIG. 11. Then, in step 120, control signals are sent by microcontroller 80 to illumination control system 83 and audio speaker 88 to produce visual and auditory cues to the user who is positioning ophthalmic instrument 10 relative to his or her eye. This process loop repeats until decision block 108 determines that the computed position difference is within the predetermined positioning tolerance for measurement purposes. When this occurs, flow branches to step 122 in which a measurement is automatically initiated, for example by energizing front coil 34 of measurement assembly 20 to propel probe P toward the eye or by activating a pump mechanism to generate a fluid pulse directed toward the eye.

The positioning system of the present disclosure provides several practical benefits for a self-measuring hand-held ophthalmic instrument. It is compact and lightweight, thereby allowing the ophthalmic instrument to be manually positioned by patients having diminished arm strength. The positioning system provides multiple sensory cues to the user to expedite the user's learning process for safely positioning the ophthalmic instrument relative to an eye and taking self-measurements. As a result, the positioning system promotes successful adoption of home use tonometry to improve patient treatment and outcomes.

While the present disclosure describes exemplary embodiments, the detailed description is not intended to limit the scope of the appended claims to the particular embodiments set forth. The claims are intended to cover such alternatives, modifications and equivalents of the described embodiments as may be included within the scope of the claims.

What is claimed is:

1. An ophthalmic instrument for measuring an ophthalmic parameter of an eye, the ophthalmic instrument comprising:
a measurement assembly operable for impinging the eye to measure the ophthalmic parameter, wherein the measurement assembly has a measurement axis for X-Y alignment with the eye to measure the ophthalmic parameter, wherein a visible portion of the measurement assembly is centered about the measurement axis when the measurement assembly is viewed by the eye along the measurement axis;
an occluder having one or more apertures collectively centered about the measurement axis; and
a source of visible light arranged to illuminate the one or more apertures with visible light;
wherein X-Y alignment of the measurement axis with the eye is indicated when the one or more apertures collectively appear concentric with the visible portion of the measurement assembly when the one or more apertures are viewed by the eye along the measurement axis.

2. The ophthalmic instrument according to claim 1, wherein the ophthalmic instrument is without a focusing optical element downstream from the source of visible light.

3. The ophthalmic instrument according to claim 1, wherein the one or more apertures is a single aperture.

4. The ophthalmic instrument according to claim 1, wherein the one or more apertures are in a plane normal to the measurement axis.

5. The ophthalmic instrument according to claim 1, wherein the visible portion of the measurement assembly has a circular profile centered about the measurement axis when the measurement assembly is viewed along the measurement axis, and the one or more apertures is a single circular aperture.

7. The ophthalmic instrument according to claim 1, wherein the measurement assembly includes a barrel enclosing one or more electromagnetic coils, wherein the measurement assembly receives a probe and propels the probe along the measurement axis toward the eye to measure the ophthalmic parameter of the eye.

8. The ophthalmic instrument according to claim 1, further comprising a translucent element arranged between the source of visible light and the occluder for diffusing light emitted by the source of visible light.

9. The ophthalmic instrument according to claim 1, wherein the source of visible light comprises an array of light-emitting diodes.

10. The ophthalmic instrument according to claim 1, further comprising an opto-electronic position detection system for automatically detecting a current X-Y-Z position of the measurement assembly relative to the eye and determining a three-dimensional position difference between the current X-Y-Z position and an ideal X-Y-Z alignment position of the measurement assembly relative to the eye.

11. The ophthalmic instrument according to claim 10, wherein the opto-electronic position detection system is connected to the source of visible light by way of an illumination control system, and wherein a color of the visible light illuminating the one or more apertures is set by the illumination control system based on the three-dimensional position difference between the current X-Y-Z position and the ideal X-Y-Z alignment position.

12. The ophthalmic instrument according to claim 11, wherein the color of the visible light illuminating the one or more apertures is set to a first color when a current Z position component of the current X-Y-Z position is closer to the eye than a tolerance range associated with an ideal Z position component of the ideal X-Y-Z alignment position.

13. The ophthalmic instrument according to claim 12, wherein the color of the visible light illuminating the one or more apertures is set to a second color different from the first color when the current X-Y-Z position is not within a tolerance range associated with the ideal X-Y-Z alignment position and the current Z position component is not closer to the eye than the tolerance range associated with the ideal Z position component.

14. The ophthalmic instrument according to claim 13, wherein the color of the visible light illuminating the one or more apertures is set to a third color different from the first and second colors when the current X-Y-Z position is within the tolerance range associated with the ideal X-Y-Z alignment position.

15. The ophthalmic instrument according to claim 13, wherein the opto-electronic position detection system is connected to the measurement assembly by way of a controller, wherein the controller causes the measurement assembly to impinge the eye when the current X-Y-Z position is within the tolerance range associated with the ideal X-Y-Z alignment position.

16. The ophthalmic instrument according to claim 14, wherein the color of the visible light illuminating the one or more apertures is set to a fourth color different from the first, second, and third colors when the opto-electronic position detection system is unable to detect the eye.

17. The ophthalmic instrument according to claim 1, further comprising a tilt sensing system for detecting if a current tilt angle of the measurement axis relative to horizontal is greater than a predetermined threshold tilt angle, wherein the tilt sensing system is connected to the source of visible light by way of an illumination control system, and wherein the illumination control system causes the source of visible light to blink on-and-off while the current tilt angle of the measurement axis is greater than the predetermined threshold tilt angle.

18. The ophthalmic instrument according to claim 10, further comprising an audio speaker connected to the opto-electronic position detection system, wherein a first sound cue is generated by the audio speaker when a current Z position component of the current X-Y-Z position is closer to the eye than a tolerance range associated with an ideal Z position component of the ideal X-Y-Z alignment position and a second sound cue different from the first sound cue is generated by the audio speaker when the current X-Y-Z position is within a tolerance range associated with the ideal X-Y-Z alignment position.

19. The ophthalmic instrument according to claim 17, further comprising an audio speaker connected to the tilt sensing system, wherein a sound cue is generated by the audio speaker while the current tilt angle of the measurement axis is greater than the predetermined threshold tilt angle.
